(19) United States
(12) Patent Application Publication
(10) Pub. No.: US 2023/0202997 A1
(43) Pub. Date: Jun. 29, 2023

(54) CHROMANOL, QUINONE OR HYDROQUINONE COMPOUNDS FOR TREATMENT OF SEPSIS

(71) Applicant: Sulfateq B.V., Groningen (NL)

(72) Inventors: Hjalmar Roland BOUMA, Groningen (NL); Guido Krenning, Groningen (NL); Robert Henk HENNING, Groningen (NL); Adrianus Cornelis VAN DER GRAAF, Groningen (NL)

(21) Appl. No.: 17/928,645

(22) PCT Filed: Jun. 2, 2021
(86) PCT No.: PCT/NL2021/050351

§ 371 Date: Nov. 30, 2022

(30) Foreign Application Priority Data
Jun. 2, 2020 (NL) 2025730

(51) Int. Cl. C07D 311/66; A61P 31/00; C07D 311/58; C07D 405/12

(52) U.S. Cl. CPC C07D 311/66; A61P 31/00; C07D 311/58; C07D 405/12

(57) ABSTRACT

The invention relates to certain chromanol, quinone or hydroquinone compounds and derivatives thereof for treatment of sepsis and sepsis-induced organ dysfunction. Specifically, the present invention relates to chromanol compounds chosen from S-(6-hydroxy-2,5,7,8-tetramethylchroman-2yl)(piperazin-1-yl)methanone and S-(6-hydroxy-2,5,7,8-tetramethylchroman-2-yl)(4-(2-hydroxyethyl)piperazin-1-yl)methanone, and pharmaceutically acceptable salts thereof.

### I. FIELD OF THE INVENTION

The invention relates to chromanol compounds and derivatives thereof for treatment or prophylaxis of sepsis. The invention further relates to chromanol compounds and derivatives thereof for the treatment or prophylaxis of sepsis-induced organ dysfunction.

### II. DESCRIPTION OF THE BACKGROUND ART

Sepsis is a deleterious systemic inflammatory response to infection. It is the major cause of morbidity and mortality worldwide (Rudd et al., Lancet 2020; 395: 200-211). Sepsis is currently defined as life threatening organ dysfunction caused by a dysregulated host response to infection. In its most severe form, sepsis causes multiple organ dysfunction that can produce a state of critical illness characterized by severe immune dysfunction and catabolism (Gotts & Matthey, BMJ 2016; 353:i1585).

The current treatment for sepsis with an emphasis on antibiotics and eradicating the source of infection, supporting blood pressure, organ blood flow, and ventilation has shown only limited efficacy in reducing mortality associated to sepsis. Despite efforts to improve current treatment strategies, the in-hospital mortality rate of sepsis in developed countries still remains around 20% (Seymour et al., N. Engl. J. Med. 2017; 376:2235-2244; Fleischmann-Struzek et al. Intensive Care Med. 2018 https://doi.org/10.1007/s00134-018-5377-4).

The sepsis response typically begins with a microbial infection. The recognition of microbial components such as lipopolysaccharide (LPS), peptidoglycan, lipoteichoic acid, and unmethylated CpG DNA by toll like receptors (TLRs) leads to the rapid activation of the innate immune response and the release of a variety of humoral mediators, including glucocorticoids, catecholamines, and proximal pro-inflammatory cytokines like tumor necrosis factor α (TNF-α), interleukin-1β (IL-1β), and IL-6. This pro-inflammatory state has been defined as being a systemic inflammatory response syndrome (SIRS).

Exaggerated production of pro-inflammatory cytokines and the induction of more distal mediators such as nitric oxide, platelet activation factor, and prostaglandins have been implicated in the endothelial changes and induction of a pro-coagulant state that leads to hypotension, inadequate organ perfusion, and necrotic cell death associated with multiple organ dysfunction syndrome (MODS).

Multiple-organ dysfunction syndrome (MODS) has been identified as one of the most fatal complications of sepsis. Many agents targeting a variety of steps in the systemic inflammatory response have been developed over the years, but most of these have shown little or no effectiveness in clinical trials.

WO 2019/172766 A1 describes the use of alkaline phosphatases for the prevention, treatment, cure, or amelioration of the symptoms of acute kidney injury caused e.g. by sepsis.

WO 2017/220810 A1 describes the use of cilastatin in treating or preventing sepsis in a mammalian subjection with the proviso that cilastatin is administered in combination with another drug which is not beta-lactam antibiotic.

U.S. Pat. No. 6,231,894 describes the use of a number of different compounds for the treatment of a disorder in which nitric oxide synthase contributes to reactive oxygen production which causes tissue injury. Most of these compounds are arginine derivatives. One of the suggested compounds is BN 80933 which is explained to be a nitric oxide synthase inhibitor that blocks electron transfer reactions of nitric oxide synthase and of NO providing compound. Compound BN 80933 has the following structure:

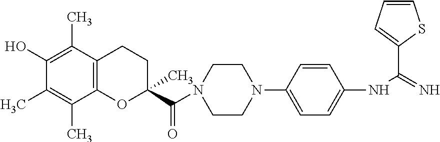 [SMILES: Cc1c(C)c2c(c(C)c1O)CC[C@@](C)(C(=O)N1CCN(c3ccc(NC(=N)c4cccs4)cc3)CC1)O2]

Although sepsis is mentioned in U.S. Pat. No. 6,231,894, the compounds do appear to be hardly effective on mitochondrial level, as shown in the experimental section below (example 5).

There remains a need for new compounds for treatment of sepsis.

It is an object of the present invention to provide compounds for the treatment or prophylaxis of sepsis, and in particular to provide compounds for the treatment or prophylaxis of organ dysfunction caused by a dysregulated host response to infection such as notably kidney dysfunction.

### III. BRIEF SUMMARY OF THE INVENTION

The above object is met by providing certain chromanol, quinone or hydroquinone compounds.

The above object is met by the present invention by providing compounds according to formula (I), (II), the hydroquinone analogue of formula (II), or a pharmaceutically acceptable salt thereof, for use in the treatment or prophylaxis of sepsis;

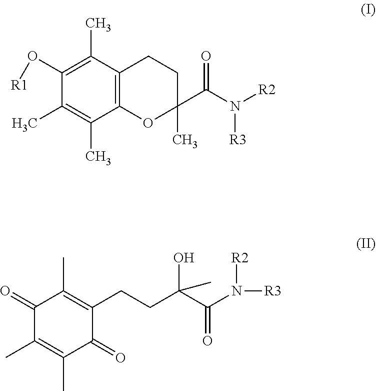

wherein R1 represents a hydrogen or prodrug moiety that can be removed in living tissue
and wherein either
   i. R2 and R3 together with the N atom to which they are attached form a saturated or unsaturated, non-aromatic, optionally substituted, 5-8 membered ring, having one to four N, O, or S atoms, wherein R2 and R3 together contain 3-12 carbon atoms;
   ii. or R2 is a hydrogen atom, or an alkyl group with 1-6 carbon atoms, and R3 is an alkyl group, optionally substituted with nitrogen or oxygen, wherein the alkyl group comprises 3-12 carbon atoms, the alkyl group in R3 comprises one or more non-aromatic cyclic structures that may comprise nitrogen or oxygen atoms in the ring, and may contain linear and/or branched substituted groups, and one or more ethylenic unsaturations;
   iii. or R2 is a hydrogen atom, or an alkyl group with 1-6 carbon atoms, and R3 is an aryl group or arylalkyl group, optionally substituted with nitrogen or oxygen, wherein R3 comprises 6-14 carbon atoms, wherein R3 comprises one or more aromatic and/or non-aromatic cyclic structures that may comprise nitrogen or oxygen atoms in the ring, and may contain linear and/or branched substituted groups, and one or more ethylenic unsaturations, wherein the compound according formula (I) or formula (II) as free base has a molecular weight lower than 400 Da.

For the present invention, the compound according to formula (II) includes the hydrogenated quinone (i.e. the hydroquinone) analogue, although the quinone derivative is preferred in view of stability.

In a preferred embodiment, the nitrogen can be amine, quaternary amine, guanidine or imine and oxygen is hydroxyl, carbonyl or carboxylic acid; and/or oxygen and nitrogen together may form amide, urea or carbamate groups.

In a preferred embodiment, R1 in formula (I) is hydrogen or forms together with the 6-oxygen an ester group with 2-6 carbon atoms.

In a preferred embodiment of either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms, such as ethylol.

In another preferred embodiment R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

In another preferred embodiment the compound is a compound according to formula II and R2 is a hydrogen atom and R3 comprises a cyclic structure having 4-6 carbon atoms and having one nitrogen atom which ring is unsubstituted or substituted with an alcohol, or alkanol group having 1-4 carbon atoms, such as ethylol, and preferably is optionally substituted with methyl, ethyl, or alcohol substituted methyl or ethyl.

In another preferred embodiment, the compound is a compound according to formula I, R2 is a hydrogen atom and R3 comprises a saturated cyclic structure having 4-7 carbon atoms and having one nitrogen atom, which ring is unsubstituted or substituted with an alcohol, or alkanol group having 1-4 carbon atoms, such as ethylol, and preferably is optionally substituted with methyl, ethyl, or alcohol substituted methyl or ethyl.

In another preferred embodiment, R3 is an aryl group or arylalkyl group, optionally substituted with nitrogen or oxygen, wherein R3 comprises 6-10 carbon atoms, wherein R3 comprises one aromatic cyclic structure that may comprise one or more nitrogen atoms in the ring, and may contain linear and/or branched aliphatic groups optionally substituted with one or two nitrogen and/or oxygen atoms.

More preferably, R2 and R3 do not comprises an aromatic ring.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

In a most preferred embodiment, the compound is the S-enantiomer of SUL-109, namely S-(6-hydroxy-2,5,7,8-tetramethylchroman-2-yl)(4-(2-hydroxyethyl)piperazin-1-yl)methanone (SUL-138) or the S-enantiomer of SUL-121, namely S-(6-hydroxy-2,5,7,8-tetramethylchroman-2yl)(piperazin-1-yl)methanone (SUL-151) or a pharmaceutically acceptable salt thereof.

In a preferred embodiment according to the invention, the compound either according to formula (I) or according to formula (II) has a molecular weight lower than 500 Da, more preferably lower than 450 Da, and even most preferred, less than 400 Da (as the free base).

In a preferred embodiment according to the invention, the compound either according to formula (I) or according to formula (II) is for use for treating or prophylaxis of sepsis in an organ system, wherein the organ is lung, heart and blood vessels, liver, kidney, brain, or intestines.

In a more preferred embodiment according to the invention, the compound the compound either according to formula (I) or according to formula (II) is for use for treating or prophylaxis of organ dysfunction caused by a dysregulated host response to infection, and in particular organ disfunction of the kidney, caused by sepsis.

The kidney appears to be a key organ in sepsis, as acute kidney injury is most frequently caused by sepsis. In turn, the occurrence of acute kidney injury is strongly associated with failure of other organs and a threefold higher in-hospital mortality rate. Moreover, acute kidney injury leads to a 9-fold increased risk for developing chronic kidney disease after sepsis, associated with an increased risk for developing end-stage renal disease. Hence, preventing or decreasing the severity of acute liver injury is one of the main goals in the treatment of sepsis, which however has to be shown to be very difficult to achieve.

The compounds for use according to the present invention, in the prophylaxis or treatment of sepsis, generally will be used as adjunct therapy, in addition to the standard therapy of antibiotics and/or other care (see Gotts, cited before).

The increased viability of organs may turn out to be very instrumental, not only in the short-term survival of sepsis, but also in the longer term. The long-term survival is of increasing importance: The case fatality rate has decreased during the last decade, which however, leads to an increased number of sepsis survivors at risk for increased long-term morbidity and in particular at major risk for (fatal) cardiovascular events. Consequently, the long-term survival rate after sepsis is less than 50% at 5 years after sepsis.

The presently invented treatment is expected to substantially improve the long-term survival rate.

### IV. SHORT DESCRIPTION OF THE FIGURES

FIG. 1 shows the xyphoid temperature of mice after the induction of sepsis with the standard cecal ligation and puncture (CLP) model, with and without SUL-138.

FIG. 2 shows the plasma levels in mice of cytokines (IL-6, TNFα, and IL12) after induction of CLP with and without treatment with SUL-138.

FIG. 3 indicates CLP-induced kidney dysfunction in mice, or the prevention thereof, depending on treatment with SUL-138, as demonstrated by measuring of NGAL and urea in plasma as biomarkers of renal function.

FIG. 4 shows CLP-induced kidney inflammation in mice, with or without treatment with SUL-138, as demonstrated by expression of RNA of a number of markers in the kidney.

FIG. 5 shows the effect of SUL-151 on survival and on geotaxis after sepsis induction in Drosophila melanogaster.

FIG. 6 demonstrates the effect of SUL-138 on LPS induced mitochondrial dysfunction and cell death in vitro.

FIG. 7 compares BN-80933, SUL-138, or SUL-150 in NO production of cellular and mitochondrial peroxide and superoxide production.

FIG. 8 compares BN-80933, SUL-138, or SUL-150 in endothelial inflammatory activation and in endothelial cell viability after hypothermia-rewarming stress.

### V. DETAILED DESCRIPTION OF THE INVENTION

The object of the present invention, to provide compounds for the treatment or prophylaxis of sepsis, and in particular to provide compounds for the treatment or prophylaxis of organ dysfunction caused by a dysregulated host response to infection such as notably kidney dysfunction, is met by providing compounds according to formula (I) or (II), as shown above, or a pharmaceutically acceptable salt thereof for use in the treatment or prophylaxis of sepsis.

In a more preferred embodiment according to the invention, the compound the compound either according to formula (I) or according to formula (II) is for use for treating or prophylaxis of organ dysfunction caused by a dysregulated host response to infection.

Organs, susceptible for damage and/or dysfunction can be one or more of lung, heart and blood vessels, liver, kidney, brain, or intestines.

The compounds according to the invention are in particular suitable to treat or prevent organ disfunction of the kidney, caused by sepsis.

The treatment or prophylaxis with the chromanol, quinone or hydroquinone compounds according to the present invention preferably is part of a combination therapy with one or more common other measures to treat sepsis.

R1 can be a substituent that is easily removed in the human body, such that the compound is a prodrug. R1 can be for example an amino acid derivative or ester derivative, and generally has a molecular weight lower than 100 dalton.

In a preferred embodiment, R1 in formula (I) is hydrogen or forms together with the 6-oxygen an ester group with 2-6 carbon atoms. The ester can comprise one or more ether or alcohol groups. Suitable esters are acetate, butyrate, 3-hydroxy butyrate and the like.

In a preferred embodiment of option (i), either compounds according to formula (I) or according to formula (II), R2 and R3 together with the N atom to which they are attached form a saturated ring having 3-6 carbon atoms and incorporating one additional N atom, which may be substituted with 1-4 carbon atoms that may comprise an oxygen, carboxylic acid or amine group.

More preferably, R2 and R3 together with the N atom to which they are attached form a 5-7 membered ring comprising one additional amine group, which ring is optionally substituted with methyl, ethyl, or alcohol substituted methyl or ethyl.

In option (i) above, R2 and R3 together have 3 or more, and 12 or less carbon atoms.

In option (ii) above, preferably R3 has 3 or more, and 12 or less carbon atoms.

In another preferred embodiment of option (ii), R2 is a hydrogen atom and R3 comprises a cyclic structure having 3-6 carbon atoms and having one nitrogen atom.

More preferably in option (ii), R2 is a hydrogen atom, and R3 comprises a 5-7 membered ring comprising one additional amine group, which ring is attached to the amide-nitrogen, and which ring is optionally substituted with methyl, ethyl, or alcohol substituted methyl or ethyl.

In either case, the ring (the cyclic structure formed by R2 and R3, or of R3 alone) may be unsubstituted or substituted with an alkyl having 1-4 carbon atoms, alcohol, or alkanol group having 1-4 carbon atoms, such as ethylol.

In another preferred embodiment according to option (iii), R3 is an aryl group or arylalkyl group, optionally substituted with nitrogen or oxygen, wherein R3 comprises not more than 10 carbon atoms. In a preferred embodiment, R3 comprises one aromatic cyclic structure that may comprise one or more nitrogen atoms in the ring, and may contain linear and/or branched aliphatic groups optionally substituted with one or two, preferably one, carboxylic acid, ester or amide groups.

In a preferred embodiment according to the invention, the compound either according to formula (I) or according to formula (II) has a molecular weight lower than 500 Da, more preferably lower than 450 Da, and even most preferred, less than 400 Da (as the free base).

In a preferred embodiment, the compound for use according the present invention is a chromanol compound according to formula I.

Certain chromanol compounds have been described in WO2014/098586. The compounds described in detail have abbreviations, referring to SUL-XXX (XXX being a 2 or 3 digit number). Many of these compounds are racemic mixtures, although some enantiomers have been tested as well. Suitable methods to prepare chromanol compounds according to the present invention are described in WO2014/098586 or WO2014/011047.

WO 2017/060432 A1 discloses amide-derivatives of 2-hydroxy-2-methyl-4-(3,5,6-trimethyl-1,4-benzoquinon-2-yl)-butanoic acid and methods of making such compounds.

Hydrogenated quinone derivatives can be easily prepared by hydrogenation of the quinone structure.

According to yet another preferred embodiment, the compound is either (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13), or (6-hydroxy-2,5,7,8-tetramethylchroman-2-yl)(4-(2-hydroxyethyl)piperazin-1-yl)methanone (SUL-109) or a pharmaceutically acceptable salt thereof, as racemic mixture or as one of its enantiomers.

In a most preferred embodiment, the compound is the S-enantiomer of SUL-109, namely S-(6-hydroxy-2,5,7,8-tetramethylchroman-2-yl)(4-(2-hydroxyethyl)piperazin-1-yl)methanone (SUL-138) or the S-enantiomer of SUL-121, namely S-(6-hydroxy-2,5,7,8-tetramethylchroman-2yl)(piperazin-1-yl)methanone (SUL-151) or a pharmaceutically acceptable salt thereof.

The counterion in the pharmaceutically acceptable salt can be a counterion as known in the art. Preferably, the compounds have at least one basic nitrogen, an amine, which can be protonated. The counterion preferably is a halogen such as chloride, sulphate, citrate, formate or the like, and most preferably chloride.

The compounds are effective as a racemic mixture or in a substantially pure enantiomeric form. The compounds have one or more chiral centers, generally one or two.

Experiments show, that for the efficacy in sepsis, the enantiomeric form is not a strongly determining factor. Nevertheless, for general regulatory reasons, preferably, the compound is a substantially enantiomerically pure compound. Substantially enantiomerically pure is about 95% enantiomeric excess or more, more preferably about 98% enantiomeric excess, and most preferably about 99% or more enantiomeric excess. Also, in case the compound contains more than one chiral center, these amounts apply.

The compounds are preferably used in effective amounts, to achieve treatment or prophylaxis of sepsis.

The wording treatment or prophylaxis includes amelioration of the symptoms of sepsis and/or reduction in progress of sepsis, including improvement of organ function.

Preferably, the compounds according to the invention are for use of treatment or prophylaxis of sepsis in organs in mammals, wherein the mammal is preferably human.

In a more preferred embodiment according to the invention, the compound the compound either according to formula (I) or according to formula (II) is for use for treating or prophylaxis of organ dysfunction caused by a dysregulated host response to infection.

In a most preferred embodiment, the compounds according to the invention are for use of treatment or prophylaxis of kidney dysfunction caused by an infection.

An infection is considered a cause from outside of the body, and is contrasted with for example autoimmune diseases. The general cause for infections is bacterial, fungal, or viral infestation. Bacterial and fungal sources are most common. A recent example of a virus causing a dysregulated host response to infection is COVID-19; upon hospital admission because of respiratory malfunction, the compounds according to the present invention may be administered prophylactically, before sepsis occurs.

Effects generally are observed with amounts of about 1 μM in body fluid, but preferably higher amounts are used. Preferred amounts are concentrations in vivo or in vitro of about 10 μM or higher, more preferably about 20 μM or higher. Generally, a concentration in human of about 200 μM or lower should be sufficient and safe.

For human use, this would mean—assuming a 30 L distribution volume, 100% availability and a concentration of about 1 μM—a dosage of about 10 mg or more. Preferred amounts would result in a concentration of about 10 μM—for which a dosage of about 100 mg or more would be suitable. Hence, preferably, dosage forms of about 20 mg or more, preferably 50 mg or more, preferably 100 mg or more are suitable.

Generally, solid, oral dosage forms contain as a maximum about 500 mg compound, preferably about 450 mg or less, to allow for excipients.

With parenteral administration, such as for example i.v., or with other liquid forms of administration, larger amounts can be administered.

Examples of dosages which can be used are an effective amount of the compounds of the invention of a dosage of 0.2 mg/kg or higher, such as preferably within the range of about 1 mg /kg to about 100 mg/kg, or within about 2 mg /kg to about 40 mg/kg body weight, or within about 3 mg/kg to about 30 mg/kg body weight, or within about 4 mg/kg to about 15 mg/kg body weight. Compounds of the present invention may be administered in a single daily dose, or the total daily dosage may be administered in divided dosage of two, three or four times daily.

The compounds described herein can be formulated as pharmaceutical compositions by formulation with additives such as pharmaceutically or physiologically acceptable excipients carriers, and vehicles.

Suitable pharmaceutically or physiologically acceptable excipients, carriers and vehicles include processing agents and drug delivery modifiers and enhancers, such as, for example, calcium phosphate, magnesium stearate, talc, monosaccharides, disaccharides, starch, gelatin, cellulose, methyl cellulose, sodium carboxymethyl cellulose, dextrose, hydroxypropyl-P-cyclodextrin, polyvinylpyrrolidone, low melting waxes, and the like, as well as combinations of any two or more thereof. Other suitable pharmaceutically acceptable excipients are described in “Remington's Pharmaceutical Sciences, ” Mack Pub. Co. , New Jersey (1991).

A pharmaceutical composition preferably comprises a unit dose formulation, where the unit dose is a dose sufficient to have a therapeutic effect. The unit dose may be a dose administered periodically in a course of treatment or suppression of a disorder.

The compounds of the invention may be administered enterally, orally, parenterally, sublingually, by inhalation (e. g. as mists or sprays), rectally, or topically in dosage unit formulations containing conventional nontoxic pharmaceutically or physiologically acceptable carriers, adjuvants, and vehicles as desired. The term parenteral as used herein includes subcutaneous injections, intravenous, intramuscular, intratarsal injection, or infusion techniques. The compounds are mixed with pharmaceutically acceptable carriers, adjuvants, and vehicles appropriate for the desired route of administration.

Generally, oral administration is a preferred route of administration, and formulations suitable for oral administration are preferred formulations.

As sepsis is often an acute disorder, oral dosage forms can be useful in cases where patients are at risk of developing sepsis, and such oral dosage forms are prophylactically administered to said patients.

However, with sepsis, in particular in acute form, iv injectables and/or continuous iv drip is preferred, because patients often are too ill for oral administration of tablets, pills or the like. Furthermore, sepsis may substantially influence the oral availability of any drug given. Certainty of plasma levels can generally only be achieved with i.v. or other parenteral administration.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

Suppositories for rectal administration of the drug can be prepared by mixing the drug with a suitable non-irritating excipient such as cocoa butter and polyethylene glycols that are solid at room temperature but liquid at the rectal temperature and will therefore melt in the rectum and release the drug.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavouring, and perfuming agents.

The amount of active ingredient that may be combined with the carrier materials to produce a single dosage form will vary depending upon the host to which the active ingredient is administered and the particular mode of administration. The unit dosage chosen is usually fabricated and administered to provide a defined final concentration of drug in the blood, tissues, organs, or other targeted region of the body. The effective amount for a given situation can be readily determined by routine experimentation and is within the skill and judgment of the ordinary clinician or skilled person.

The present invention will be further illustrated using the examples below. In the examples, reference is made to figures.

### VI. EXAMPLES

The effectiveness of the compounds according to the invention for treatment or prophylaxis of sepsis was tested in vivo in mice and in drosophila, and in vitro in HUVA, HUVEC or NRK cells. (examples 1-4)

### Example 1; Mice

Experimental

The animal experiments were approved by the Institutional Animal Care and Use Committee of the University Medical Center Groningen (IvD nr. 16593).

Male C57/BL6J mice were housed at room temperature at a light-dark cycle of 12:12 hour. Animals were fed ad libitum using standard animal lab chow and they had free access to drinking water at all times.

To induce sepsis, the standard cecal ligation and puncture (CLP) model was used. Animals were anesthetized by subcutaneous injection of xylazine/ketamine (100/10 mg/kg), followed by administration of buprenorphine (0.1 mg/kg) as analgesic. After confirmation of anesthesia by lack of response to paw pinch and eye reflex, the abdomen was shaved, cleaned, and de-germed using a povidone-iodine solution before a 1-cm midline incision was made. The cecum was ligated with a 6-0 suture at half the distance between distal pole and the base of the cecum and punctured once with a 21-Gauge needle (‘through-and-through’ from mesenteric toward anti-mesenteric direction) which is an accepted model for ‘mid-grade’ sepsis. A small amount of stool (2-3 mm) was then extruded to ensure wound patency. The cecum was repositioned, thereby taking care not to spill fecal material on the wound edges, followed by closure of the abdomen by running sutures to the abdominal musculature and short interrupted sutures to the skin. Next, 1 ml of saline (warmed, 0.9% NaCl s.c.) was administered to compensate for the expected relative volume depletion due to the onset of sepsis.

Mice recovered at 26-28° C. Broad-spectrum antibiotics (imipenem/cilastatine, 100 mg/kg s.c.) were administered at 2 and 10 hours following surgery, together with analgesics (buprenorphine, 0.1 mg/kg body weight, s.c.).

A group of operated animals, in which the cecum was located but not punctured, served as sham.

In addition, a group of animals was included that underwent time-matched anesthesia, but no surgery and served as controls.

Mice in the SUL-138 treated groups were injection with SUL-138 (dissolved in saline, 5 mg/kg, s.c.) at 2 hours before and 8 hours after surgery, while mice in the other groups were injected with an equal volume of saline at these time-points.

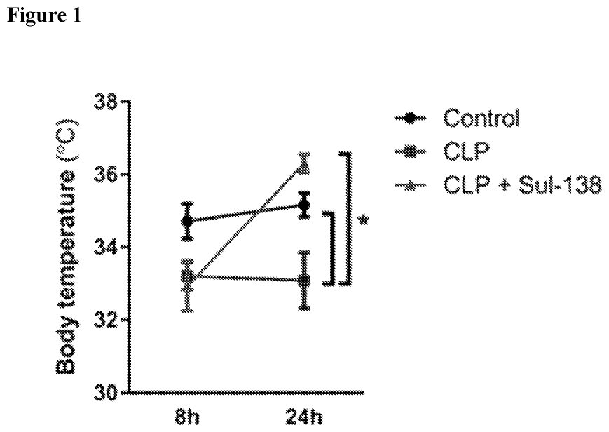

The xyphoid temperature of the mice was measured at 8 hr and 24 hr after procedure; results are shown in FIG. 1, discussed below.

Mice were sacrificed 24 hours after the procedure. Upon euthanization, EDTA-anticoagulated blood was separated into plasma by centrifugation at 1,600 g for 10 min and serum by allowing it to clot for 30 min followed by centrifugation at 3,000 g for 10 min.

Plasma, serum and organs were snap-frozen in liquid nitrogen for further analysis.

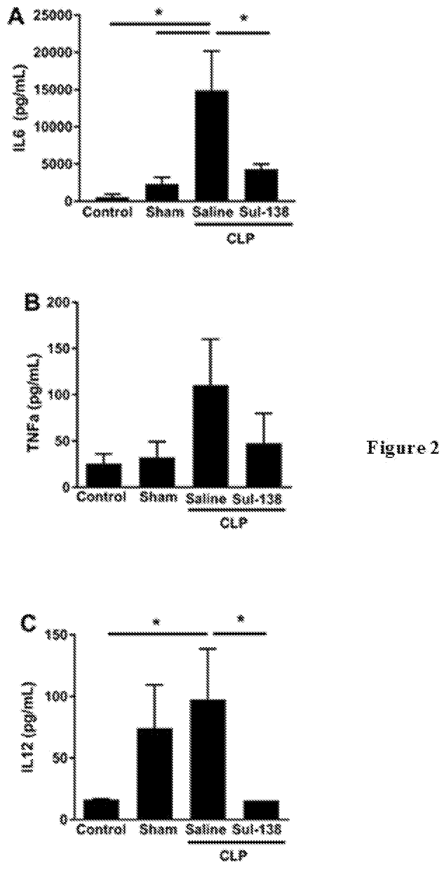

To quantify the effect of sepsis severity and treatment with SUL-138 on systemic inflammation, levels of TNFα, IL-6 and IL-12 in plasma were measured using Mouse DuoSet ELISAs (DY410, DY406 and D419, respectively, RnD-Systems), according to the manufacturer's instructions. Briefly, ELISA plates (DY990, RnD Systems) were coated overnight with the capture antibody diluted in 100 μL PBS. Plates were washed three times with wash buffer (0.05% Tween20 in PBS; 137 mM NaCl, 2.7 mM KCl, 8.1 mM Na2HPO4, 1.5 mM KH2PO4, pH 7.2-7.4), followed by blocking for one hour with 300 μL of reagent diluent (1% probumin w/v in PBS). Washing was repeated and samples were added to the wells. Plasma samples were diluted 10× for TNFα and IL-12, and 100× for IL-6 in reagent diluent. After incubation for 2 hours at room temperature, plates were washed, followed by adding 100 μL of detection antibody diluted in reagent diluent to each well. Again, plates were left to incubate for 2 hours at room temperature, followed by washing. Finally, 100 μL of substrate solution (DY999, RnD Systems) was added and after 20 minutes incubating in the dark, 50 μL stop solution (2M H2SO4) was added. The optical density (OD) was measured using a microplate reader set to 450 nm, while readings at 540 nm were subtracted as correction to increase accuracy. Results are shown in FIG. 2, discussed below.

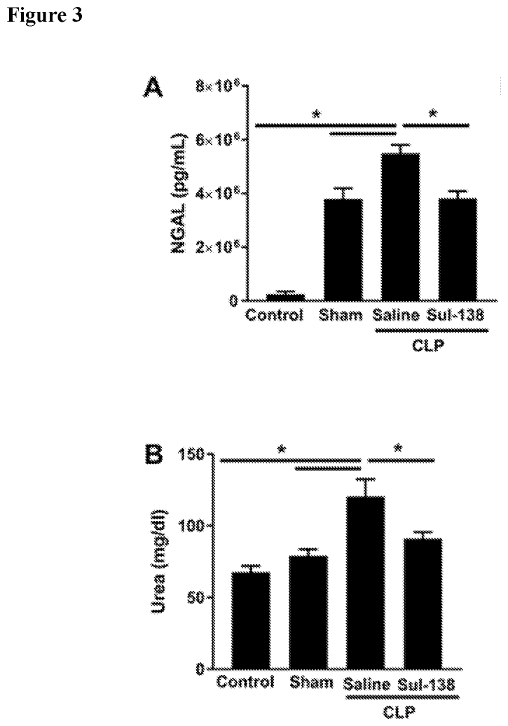

Furthermore, the amount of NGAL and urea were measured in serum. NGAL and urea are common biomarkers for kidney damage in mice; results are shown in FIG. 3, discussed below.

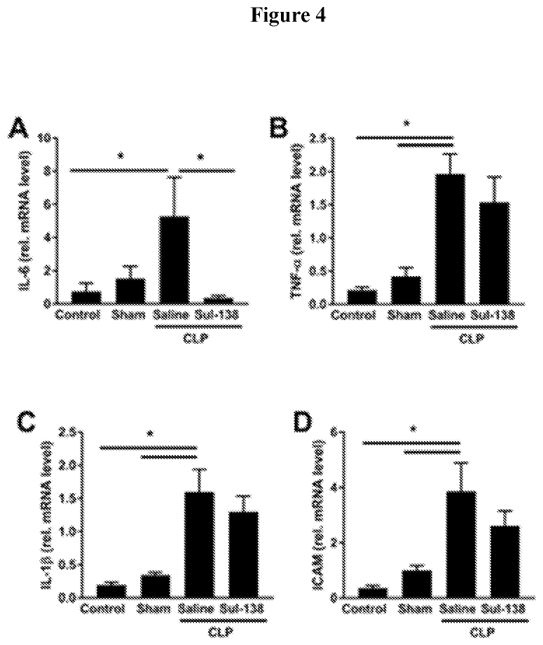

In order to assess the expression of proinflammatory cytokines and adhesion molecules in the kidney, including IL-6, TNF-α, IL-1β and ICAM, RNA was isolated from approximately 30 mg kidney tissue using Nucleospin RNA (Machery-Nagel, Duren, Germany), quantified using nanodrop spectrophotometer ND-1000 and converted into copy DNA (cDNA), using 0.5 μg RNA in each sample. For RNA isolation from cells, the same kit was used with slight adaptations: TRIzol and chloroform as lysis buffer, instead of the lysis buffer from the kit. Olignucleotide primers were designed using NCBI Primer Blast and Clone Manager (see appendix) and validated by assessing the efficiency, melting temperature and curve using qRT-PCR and size on gel electrophoresis of the naive and enzymatically digested products. qRT-PCR amplification was performed using the following thermal profile: 95° C. for 2 min, followed by 40 cycli of 95° C. for 15 sec, 58° C. for 30 sec and 72° C. 30 sec. All reactions were carried out in triplicate and a standard curve for each primer was used. Results are shown in FIG. 4, discussed below.

Results

FIG. 1 shows the xyphoid temperature of mice after the induction of CLP with and without SUL-138 versus an unchallenged control. FIG. 1 demonstrates that CLP procedures result in a reduction of xiphoid temperature after 8 hr and 24 hr, while upon treatment with SUL-138 xiphoid temperature is restored after 24 hours. Significant differences are calculated at 24 hr between unchallenged control versus CLP/saline and CLP/saline versus CLP/SUL-138 using an unpaired one-sided Student's T-test. A ‘*’ in the figures indicates a significant difference.

FIG. 2 shows that the plasma levels of the inflammatory cytokines (IL-6, TNF-α and IL12) are decreased by treatment with SUL-138 to levels close to untreated control. Significant differences are calculated between control versus CLP/saline, Sham versus CLP/saline and CLP/saline versus CLP/Sul-138 using an unpaired one-sided Student's T-test. TNF-α was clearly lower, but the difference did not reach significance in the T test.

FIG. 3 indicates that CLP-induced kidney dysfunction is precluded by treatment with SUL-138. Serum urea (A) and NGAL (B) levels are profoundly increased in sepsis, which is precluded by treatment with SUL-138. Serum urea and NGAL levels in animals treated with SUL-138 before CLP were not different from Sham operated animals. Significant differences are calculated between control versus CLP/saline, Sham versus CLP/saline and CLP/saline versus CLP/SUL-138 using an unpaired one-sided Student's T-test.

FIG. 4 indicates that CLP-induced kidney inflammation is strongly reduced by treatment with SUL-138. Sepsis induced by CLP upregulated the expression of proinflammatory cytokines and adhesion molecules in the kidney, including IL-6, TNF-α, IL-1β and ICAM. Treatment of animals before induction of sepsis by CLP with SUL-138 fully prevented the rise in IL-6 expression (see FIG. 4A). TNF-α, IL-1β or ICAM (FIG. 4B-D) were lower than in the untreated CLP animals but the difference was not statistically significant using an unpaired one-sided Student's T-test.

Discussion

The reduced body temperature of mice after induction of sepsis illustrates loss of metabolic homeostasis, the restoration after 24 h in mice treated with SUL-138 suggests restoration of metabolic homeostasis despite sepsis.

The increased plasma levels of IL-6 and IL-12 indicate systemic inflammation induced by CLP, while both cytokines are significantly lower after CLP in animals treated with SUL, indicative for reduced levels of inflammation.

The increased levels of NGAL and urea after induction of CLP indicate acute kidney dysfunction. NGAL and urea are biomarkers for renal function in mice. FIG. 3 shows that CLP-induced kidney dysfunction in mice is precluded, or at least substantially reduced by treatment with SUL-138.

RNA expression of IL-6, TNF-α, IL-1β and ICAM in the kidney were increased after induction of CLP, indicating a local inflammatory response in the kidney. Treatment with Sul-138 significantly reduced expression of IL-6, while other markers were lowered by treatment with SUL-138.

### Example 2; Drosophila melanogaster

Experimental

W1118 flies were bred and housed at 25° C. with a 12 h:12 h light/dark-cycle. Flies were kept in vials containing approximately 5 mL of standard yeast-cornmeal medium. Flies kept as a stock were flipped to fresh vials weekly. The used yeast-cornmeal medium was prepared to the instructions of Stocker and Gallant's ‘Drosophila Methods and Protocols’ and consisted of 100 grams yeast, 75 grams glucose, 8 grams agar, 55 grams cornmeal, and 10 grams wheat flour per litre of water. After boiling, phosphoric acid and propionic acid were added to the mixture to prevent fungal growth.

Staphylococcus aureus (S. aureus) was aerobically cultured overnight in 2.5% tryptic soy broth (TSB) at 37° C. with continuous rotation (200 rounds per minute), from an S. aureus glycerol stock kept at −80° C. For each experiment, a fresh culture was prepared one day prior to the experiment. For bacterial infection, optical density (OD) was measured the following day at 600 nm. Clean TSB was used as OD=0.00 and the bacterial culture was diluted to OD=2.20 with PBS. Next, 1.0 mL of the bacterial culture (OD=2.20) was centrifuged at 14,000 g for 1 minute, the supernatant was discarded, and the bacterial pellet resuspended in 1.0 mL PBS. Serial dilutions (5× and 25× diluted in PBS with optical densities of approximately 1.05 and 0.25, respectively) were used in the appropriate experiments.

Male flies (three to five days old) were anesthetized on a CO₂ pad just prior to injection. A tungsten needle (0.25 mm diameter, Fine Science Tools, 10130-10) was dipped into the bacterial suspension and flies were pinpricked in the lateral side of the thorax (the presutural scutum of the thorax). Sham flies were administered PBS only, control flies were not operated and only anesthetized on the CO₂ pad. Sham flies were injected first to ensure a haemolymph coating on the needle and to prevent accidental bacterial injection of sham flies. Subgroups of flies were treated with antibiotics. Therefore, linezolid was administered orally by dissolving 500 μg of the substance per 1 mL fly medium. After pinpricking intervention groups with various concentrations of S. aureus, flies were placed in vials containing linezolid-medium.

After 48 hours, the flies were introduced to non-antibiotic containing vials. To verify the efficiency in inducing a bacterial infection, bacterial load was determined by homogenization of ten flies in 250 μL PBS. After brief centrifugation, the supernatant was plated in 1/10, 1/100, and 1/1000 dilutions onto Luria-Bertani (LB) agar plates and checked 24 hours later.

Linezolid was administered orally by dissolving 500 μg of the substance per 1 mL fly medium. After pinpricking, control flies, sham-operated flies and the infected intervention groups with various concentrations of S. aureus, flies were placed in vials containing linezolid-medium. After 48 hours, the flies were introduced to non-antibiotic containing vials.

The compound SUL-151 was administered via intrathoracic injection simultaneously with the bacterial injection. The intervention group received 3 mM SUL-151 and the vehicle infected flies received 1% DMSO, which were dissolved in the bacterial solution itself just prior to bacterial injection.

Negative geotaxis was studied as a marker of fly health. Therefore, groups of 15 flies were transferred to empty styrene vials (9.5 cm tall). Up to nine vials were then placed into a 3d-printed vial holder designed for negative geotaxis. Flies were allowed to acclimatize for five minutes. The flies were then tapped to the bottom of the vial by tapping the vial holder three times against the worktop. A digital camera was used to take a picture five seconds after the last tap. This experiment was repeated five times, with a one-minute pause between every try. ImageJ was used to determine the distance every fly travelled within five seconds and averaged over five tries per group.

Additionally, fly survival was checked by visual inspection, counting dead flies in the vial, at 24 hours after infection.

Results

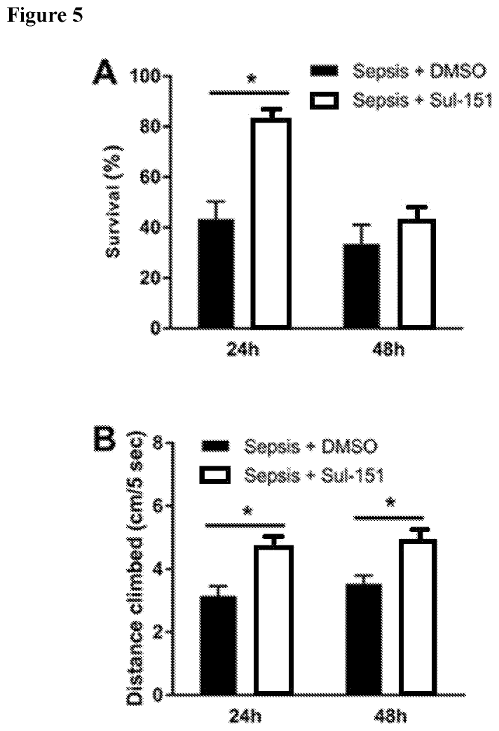

FIG. 5 shows the effect of SUL-151 on survival (A) and geotaxis (B) after sepsis induction in Drosophila melanogaster. Survival after 24 hr was 80% for SUL-151 treated flies, while only 42% for the flies with sepsis without SUL treatment (A). Geotaxis was improved both at 24 hr and 48 hr after induction of sepsis in flies pre-treated with SUL (B). Significant differences are calculated between control versus sepsis/DMSO and sepsis/DMSO versus sepsis /SUL-151 using an unpaired one-sided Student's T-test.

Discussion

Injection of 3 mM SUL-151 at the induction of sepsis lead to improved geotaxis at 24 and 48 h after the induction of sepsis, while mortality was significantly reduced at 24 hr. Both measures indicate efficacy of SUL151 against infection induced inflammation.

### Example 3; Endothelial Cells

Experimental

Human Umbilical Vein Endothelial cells (HUVEC) were obtained from the RuG/UMCG Endothelial Cell Facility. Primary isolates of umbilical cords were mixed and subsequently cultured on HUVEC culture medium, consisted of RPMI 1640 (Lonza, art.nr. BE12-115F) supplemented with 20% heat-inactivated fetal calf serum (ThermoFisher Scientific, art.nr. 10082147), 2 mM 1-glutamine (Life Technologies art.nr. 25030), 5 U/ml heparin (Leo Pharmaceutical Products), 1% Penicillin/Streptomycin (Sigma-Aldrich art.nr. P4333), and 50 μg/ml EC growth factor supplement from (Sigma-Aldrich, art.nr. E2759).

Primary HUVECs were cultured in 75-cm2 tissue culture flasks (Corning, art.nr. 430720U) at 37° C. under 5% CO2/95% air. HUVECs were used for experiments up to passage 8. Experiments were performed in 6-well (Corning art.nr. 3506) or 96-well culture plates (Corning, art.nr. 3596), at 80% confluency. Cells were stimulated with LPS E. coli 0111:B4 (Sigma-Aldrich, art.nr. L2630) in different concentrations. Cells were detached with trypsin (Sigma-Aldrich, art.nr. 25300054). All compounds were dissolved in Hanks Balanced Salt Solution (Lonza, art.nr. 10-527F).

HUVECs were pre-incubated with SUL-138 in an amount of 10 microgram/ml, which was added one hour prior to LPS-stimulation. Such pre-incubation is the standard in in vitro models.

Membrane potential was measured by using JC-1 (5,5′,6,6′-tetrachloro-1,1′,3,3′-tetraethyl-imidacarbocyanine iodide). Mitochondrial membrane potential was measured after 30 minutes.

Cells and mitochondria were incubated and measured according to manufacturer's protocol in the Synergy H4 micro plate reader (Bio-Tek) at an excitation/emission rate of 548/574 nm.

Results

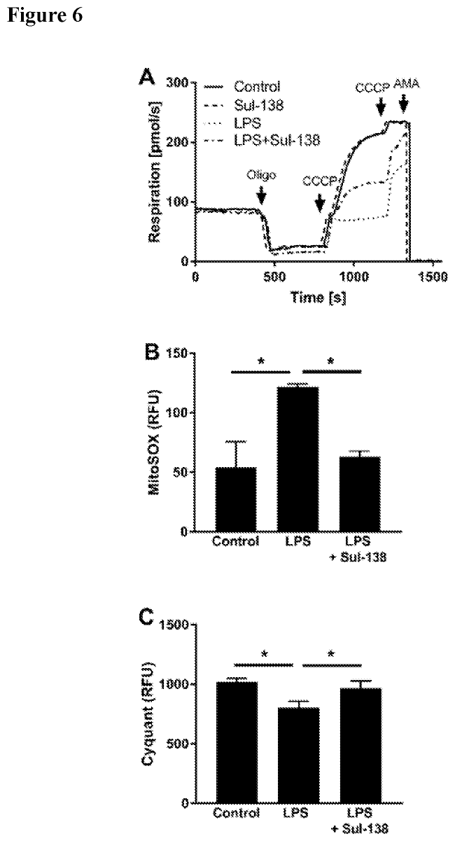

FIG. 6 demonstrates that SUL-138 protects against LPS induced mitochondrial dysfunction and cell death in vitro.

FIG. 6A demonstrates the HUVECs respiration and shows a reduction in uncoupled state (CCCP) with 10 ug/ml LPS, compared to control. LPS challenged HUVECs treated with SUL-138 almost restores the uncoupled respiration.

FIG. 6B shows the LPS-induced increased mitochondrial oxidative stress, measured by MitoSOX. MitoSOX measures ROS production in the mitochondrial, thus SUL-138 reduces LPS induced mitochondrial oxidative stress.

FIG. 6C demonstrates that 48 hours with LPS on HUVECs resulted in reduced cell survival, while SUL-138 restores cell survival after 48 hours. Cell death was measured by Cyquant.

Significant differences are calculated between control versus LPS and LPS versus LPS/SUL-138 using an unpaired one-sided Student's T-test.

Discussion

The experiments performed on the LPS-treated endothelial cells demonstrate that sepsis on the cellular level involves mitochondrial dysfunction, which is restored by compounds according to the invention, while whole cell survival increased significantly with the use of SUL-138.

Without being bound by theory, the inventors believe that the compounds according to the invention can be used for the treatment or prophylaxis of sepsis because they protect the cells against LPS-induced mitochondrial dysfunction and cell death.

### Example 4 (In Vitro Tests with Several SUL Compounds)

The following compounds were tested:

| | | | | |
|---|---|---|---|---|
| Compound | Chemical Name | Formula | Structure | MW |

Compounds not according the invention

| | | | | |
|---|---|---|---|---|
| SUL-89 | 6-hydroxy-2,5,7,8-tetramethyl- 3,4-dihydro-2H-1-benzopyran- 2-carboxamide | C₁₄H₁₉NO₃ | 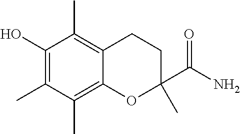 [SMILES: Cc1c(C)c2c(c(C)c1O)CCC(C)(C(N)=O)O2] | 249.3 |
| SUL-99 | methyl [(6-hydroxy-2,5,7,8- tetramethyl-3,4-dihydro-2H- 1-benzopyran-2- carbonyl)amino]acetate | C₁₇H₂₃NO₅ | 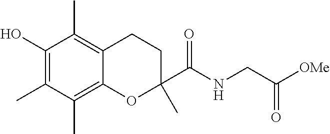 [SMILES: COC(=O)CNC(=O)C1(C)CCc2c(C)c(O)c(C)c(C)c2O1] | 321.4 |
| SUL-127 | methyl 6-hydroxy-2,5,7,8- tetramethyl-3,4-dihydro-2H- 1-benzopyran-2-carboxylate | C₁₅H₂₀O₄ | 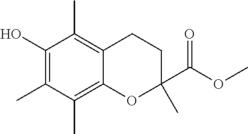 [SMILES: COC(=O)C1(C)CCc2c(C)c(O)c(C)c(C)c2O1] | 264.3 |

Compounds according the invention

| | | | | |
|---|---|---|---|---|
| SUL-13 | (2S)-6-hydroxy-2,5,7,8-tetra- methyl-N-((R)- piperidin-3-yl)-3,4-dihydro-1- benzopyran-2-carboxamide | C₁₉H₂₈N₂O₃ | 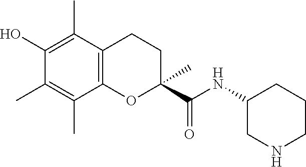 | 332.4 |
| SUL-96 | N-benzyl-6-hydroxy-2,5,7,8- tetramethyl-3,4-dihydro-2H-1- benzopyran-2-carboxamide | C₂₁H₂₅NO₃ | 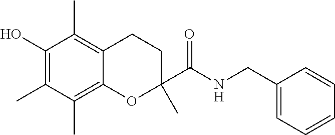 [SMILES: Cc1c(C)c2c(c(C)c1O)CCC(C)(C(=O)NCc1ccccc1)O2] | 339.4 |
| SUL-103 | 6-hydroxy-2,5,7,8-tetramethyl- N-phenyl-3,4-dihydro-2H-1- benzopyran-2-carboxamide | C₂₀H₂₃NO₃ | 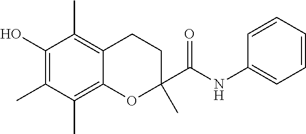 [SMILES: Cc1c(C)c2c(c(C)c1O)CCC(C)(C(=O)Nc1ccccc1)O2] | 325.4 |
| SUL-129 | 6-hydroxy-N-[(1S)-2-hydroxy- 1-phenylethyl]-2,5,7,8-tetra- methyl-3,4-dihydro-2H-1- benzopyran-2-carboxamide | C₂₂H₂₇NO₄ | 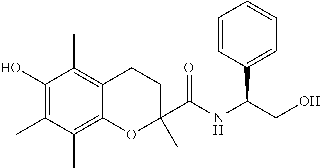 [SMILES: Cc1c(C)c2c(c(C)c1O)CCC(C)(C(=O)N[C@H](CO)c1ccccc1)O2] | 355.5 |
| SUL-138 | (S)-(6-hydroxy-2,5,7,8- tetramethylchroman-2-yl)(4- (2-hydroxyethyl)piperazin-1- yl)methanone | C₂₀H₃₀N₂O₄ | 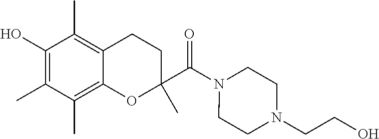 [SMILES: Cc1c(C)c2c(c(C)c1O)CCC(C)(C(=O)N1CCN(CCO)CC1)O2] | 362.5 |
| SUL-138M2 | 4-(2,5-dihydroxy-3,4,6- trimethylphenyl)-2-hydroxy- 1-(4-(2- hydroxyethyl)piperazin-1-yl)- 2-methylbutan-1-one | C₂₀H₃₂N₂O₅ | 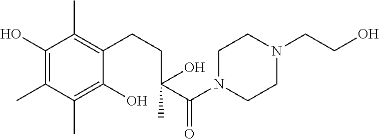 [SMILES: Cc1c(C)c(O)c(CC[C@@](C)(O)C(=O)N2CCN(CCO)CC2)c(C)c1O] | 380.5 |
| SUL-150 | (R)-(6-hydroxy-2,5,7,8- tetramethylchroman-2- yl)(piperazin-1-yl)methanone | C₁₈H₂₆N₂O₃ | 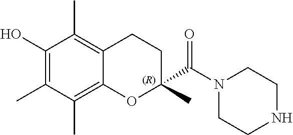 | 318.4 |
| SUL-151 | (S)-(6-hydroxy-2,5,7,8- tetramethylchroman-2- yl)(piperazin-1-yl)methanone | C₁₈H₂₆N₂O₃ | 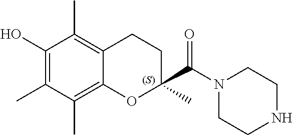 | 318.4 |

Experimental Design

HUVEC Cell Culture

Human Umbilical Cord Endothelial Cells (HUVEC, Lonza CC-2519) were maintained on 1% gelatin-coated culture flasks in RPMI1640 medium containing 20% fetal bovine serum, 2 mM Glutamine (Sigma-Aldrich), 1% Penicillin-Streptomycin solution (Sigma-Aldrich) and 50 μg/ml Bovine Pituitary Extract (Invitrogen). HUVEC were passaged by trypsinization when the cultures reach a confluency of 70%. For all experiments, HUVEC were seeded at 0.6·10⁵ cells/cm² and allowed to adhere for 24 hours.

NRK52E Cell Culture

Rat kidney epithelial cells (ATCC #CRL-1571) were maintained in DMEM medium containing 10% fetal bovine serum, and 1% Penicillin-Streptomycin solution (Sigma-Aldrich). NRK52E cells were passaged by trypsinization when the cultures reached a confluency of 70%. For all experiments, NRK52E cells were seeded at 0.6·10⁵ cells/cm² and allowed to adhere for 24 hours.

Inflammatory Activation After Endotoxin Exposure

HUVEC and NRK52E cells were pre-incubated with SUL compounds (10 μM) for 30 min and subsequently exposed to LPS for 24 hours under standard culture conditions. Cells were lysed in triZOL reagens (Invitrogen) and total RNA isolated according to manufacturers' instruction. 1 μg total RNA per sample was reverse transcribed using the FirstAid Reverse Transcription Kit (ThermoFisher). Copy-DNA equivalents of 5 ng total RNA were amplified on a ViiA7 Real-time PCR system (ThermoFisher, Waltham, Mass.) using iTaq Universal SYBR Green Supermix (Bio-Rad, Hercules, Calif.) and primers specific for tumor necrosis factor alpha, interleukin 1 beta, interleukin-6 and beta actin as loading control. Amplification was performed for 40 cycles of 95° C. for 30 s, 60° C. for 1 min. Relative mRNA expression was calculated as 2^{-(Cq}^{_{gene}}^{-Cq}^{_{reference}}⁾ and normalized to unstimulated control cells (100% inhibition) and vehicle-treated and LPS exposed cells (0% inhibition). The following primers were used:

qPCR primers

| | |
|---|---|
| Sense primer | Antisense primer |

Human PCR primers

| | | |
|---|---|---|
| TNFα | 5′-CAGCCTCTTCTCCTTCCTGAT-3′ | 5′-GCCAGAGGGCTGATTAGAGA-3′ |
| IL-1β | 5′-AAGCTGGAATTTGAGTCTGC-3′ | 5′-ACACAAATTGCATGGTGAAG-3′ |
| IL-6 | 5′-AGCTCAATAAGAAGGGGCCTA-3′ | 5′-TGAGAAACCCTGGCTTAAGTAGA-3′ |
| ACTB | 5′-CCAACCGCGAGAAGATGA-3′ | 5′-CCAGAGGCGTACAGGGATAG-3′ |

Rat PCR primers

| | | |
|---|---|---|
| TNFα | 5′-ATGGGCTCCCTCTCATCAGT-3′ | 5′-GCTTGGTGGTTTGCTACGAC-3′ |
| IL-1β | 5′-CAGCTTTCGACAGTGAGGAGA-3′ | 5′-TTGTCGAGATGCTGCTGTGA-3′ |
| IL-6 | 5′-TTTCTCTCCGCAAGAGACTTCC-3′ | 5′-TCTCCTCTCCGGACTTGTGAA-3′ |
| ACTB | 5′-GTTGCGCGTGCGTATTGAG-3′ | 5′-CGCAGGACAGCCGCATTAT-3′ |

Statistical Evaluation

All experiments were performed minimally in triplicate per condition. Data obtained from individual experiments were used for evaluation in GraphPad Prism 9.0 (GraphPad Software Inc, Ca). ANOVA followed by Bonferroni post hoc analysis was used to calculate statistical significance. Probability values (p) below 0.05 were considered significant.

Results

Endothelial and Epithelial Inflammatory Activation.

Endothelial inflammatory activation plays an important role in modulating inflammatory processes during sepsis. Triggered by endotoxins or other pro-inflammatory molecules, endothelial cells start to produce inflammatory cytokines, which subsequently mobilize and recruit inflammatory cells resulting in an inflammatory response.

Endothelial and kidney epithelial cells have a very low baseline gene expression for inflammatory cytokines, which is strongly increased upon exposure to endotoxins such as LPS. Pre-treatment of endothelial or kidney epithelial cells with SUL compounds (all 10 μM for 30 min), prior to an LPS challenge precludes the induction of mRNA expression of the pro-inflammatory cytokine TNFα and the interleukins IL-1β and IL-6 with varying efficiency, as shown in the following table:

Inhibition of LPS-induced inflammatory cytokine expression by SUL compounds.

| | HUVEC | | | NRK52E | | |
|---|---|---|---|---|---|---|
| | TNFα | IL-1β | IL-6 | TNFα | IL-1β | IL-6 |
| Vehicle | 100 ± 0.8 | 100 ± 0.5 | 100 ± 0.4 | 100 ± 0.8 | 100 ± 2.7 | 100 ± 0.3 |
| (unstimulated) | | | | | | |
| Vehicle (LPS) | 0.0 ± 2.6 | 0.0 ± 4.3 | 0.0 ± 1.8 | 0.0 ± 2.8 | 0.0 ± 4.5 | 0.0 ± 10.1 |
| SUL-89 | — | — | — | 17.5 ± 3.4 | 12.3 ± 6.4 | 8.7 ± 4.6 |
| SUL-99 | 35.1 ± 6.2* | 31.5 ± 4.5* | 32.7 ± 8.1* | | | |
| SUL-127 | 28.6 ± 3.2 | 26.7 ± 3.1 | 22.2 ± 7.2 | 29.1 ± 7.6 | 19.6 ± 6.1 | 21.2 ± 7.2 |
| SUL-13 | 41.2 ± 8.2* | 38.6 ± 4.2* | 45.7 ± 9.1* | | | |
| SUL-96 | — | — | — | 85.4 ± 4.7* | 81.7 ± 7.2* | 89.9 ± 5.8* |
| SUL-103 | — | — | — | 67.8 ± 7.8* | 59.2 ± 7.9* | 63.3 ± 1.4* |
| SUL-129 | — | — | — | 75.6 ± 6.4* | 75.9 ± 7.1* | 69.2 ± 2.6* |
| SUL-138 | 81.4 ± 4.5* | 76.3 ± 7.9* | 68.3 ± 6.7* | 52.6 ± 11.6* | 65.2 ± 8.9* | 65.3 ± 7.6* |
| SUL-138M2 | 58.1 ± 3.1* | 51.7 ± 8.0* | 55.2 ± 8.5* | — | — | — |
| SUL-150 | 79.8 ± 4.1* | 78.9 ± 6.0* | 69.3 ± 3.9* | — | — | — |
| SUL-151 | 72.5 ± 3.2* | 76.7 ± 1.2* | 65.4 ± 4.6* | 72.1 ± 14.5* | — | 83.1 ± 8.3* |

Data indicate percentage (%) inhibition of LPS-induced mRNA expression of TNFα, IL-1β or IL-6 after normalization to unstimulated vehicle-treated control cells (100% inhibition) and LPS-stimulated vehicle-treated control cells (0% inhibition). All experiments were performed in triplicate per cell line and SUL compound.
— = not determined; *p < 0.05.

From this table it is apparent that compounds not according the invention show a percentage inhibition of about 35% or less, while compounds according the invention show inhibition of about 40% or more.

Furthermore, a comparison of SUL-150 and SUL-151 shows that the effect is not dependent on the enantiomeric configuration. Comparison of SUL-138 and SUL-138M2 shows that compounds according to formula 2 are less preferred, but also show activity.

### Example 5 and Comparative Experiment A

In this study, the mechanism of action of the SUL compounds SUL-138 and SUL-150 (according to the invention) was compared to the mechanism of action of BN-80933 (comparative A, structure see introduction above) in a range of in vitro experiments focused on the release of NO, inhibition of cytoplasmic and mitochondrial oxidative stress, inflammatory signaling, and cell survival after hypothermia-rewarming stress. These in vitro assay have been used prior to establish the efficacy of named compounds and are therefore considered proper indicators of their mechanism of action.

Experimental Design

HUVEC Cell Culture

Human Umbilical Cord Endothelial Cells (HUVEC, Lonza CC-2519) were maintained on 1% gelatin-coated culture flasks in RPMI1640 medium containing 20% fetal bovine serum, 2 mM Glutamine (Sigma-Aldrich), 1% Penicillin-Streptomycin solution (Sigma-Aldrich) and 50 μg/ml Bovine Pituitary Extract (Invitrogen). HUVEC were passaged by trypsinization when the cultures reach a confluency of 70%. For all experiments, HUVEC were seeded at 0.6·10⁵ cells/cm² and allowed to adhere for 24 hours.

RAW264.7 Cell Culture

Mouse macrophage RAW264.7 cells (ATCC #TIB-71) were maintained in DMEM medium containing 10% fetal bovine serum, and 1% Penicillin-Streptomycin solution (Sigma-Aldrich). RAW264.7 cells were passaged by trypsinization when the cultures reached a confluency of 70%. For all experiments, RAW264.7 cells were seeded at 0.5·10⁵ cells/cm² and allowed to adhere for 24 hours.

Determination of NO Production

Cellular production of NO was approximated by measuring nitrate concentrations in culture media using the Measure-IT™ High-Sensitivity Nitrite Assay Kit (#M36051, ThermoFisher) according to manufacturer's instructions. Produced NO is unstable with a half-life of 2-30 s and rapidly reacts with molecular oxygen to form nitrite, which in cell culture media is further oxidized to nitrate. Therefore, nitrate concentrations in conditioned media are a good approximate to assess cellular NO production. 50 μl conditioned medium derived from a single 96-wells plate well was used per assay and normalized against protein concentration. All experiments were performed in quadruplicate.

Determination of LPS-Induced Cellular and Mitochondrial Oxidative Stress.

The production of cellular and mitochondrial oxidative stress was measured using fluorescent probes for cellular and mitochondrial superoxide, i.e. dihydroethidium (DHE) and MitoSox (ThermoFisher), respectively. HUVEC were incubated with BN-80933, SUL-138 or SUL-150 (all 10 μM) for 30 min. Next, HUVEC were exposed to LPS (100 ng/ml) for an additional 24 hours. In the final hour of incubation, 2.5 μM of DHE or MitoSox was applied to each sample. HUVEC were washed in PBS and fixed in 4% paraformaldehyde at room temperature for 15 min. Nuclear staining was performed by 5 μM 2-(4-amidinophenyl)-1H-indole-6-carboxamidine (DAPI) and fluorescence intensity of the samples was recorded in a CLARIOStar Plus plate reader (BMG Labtech) equipped with the appropriate filter sets. Fluorescence recordings for DHE and MitoSox were corrected for DAPI fluorescence. All experiments were performed in quadruplicate.

Inflammatory Activation After Endotoxin Exposure

HUVEC cells were lysed in triZOL reagens (Invitrogen) and total RNA isolated according to manufacturers' instruction. 1 μg total RNA per sample was reverse transcribed using the FirstAid Reverse Transcription Kit (ThermoFisher). Copy-DNA equivalents of 5 ng total RNA were amplified on a ViiA7 Real-time PCR system (ThermoFisher, Waltham, Mass.) using iTaq Universal SYBR Green Supermix (Bio-Rad, Hercules, Calif.) and primers specific for tumor necrosis factor alpha (TNFα; sense 5′-CAGCCTCTTCTCCTTCCTGAT-3′, antisense 5′-GCCAGAGGGCTGATTAGAGA-3′), interleukin 1 beta (IL-1β; sense 5′-AAGCTGGAATTTGAGTCTGC-3′, antisense 5′-ACACAAATTGCATGGTGAAG-3′), interleukin-6 (IL-6; sense 5′-AGCTCAATAAGAAGGGGCCTA-3′, antisense 5′-TGAGAAACCCTGGCTTAAGTAGA-3′) and beta actin (ACTB; sense 5′-CCAACCGCGAGAAGATGA-3′, antisense 5′-CCAGAGGCGTACAGGGATAG-3′) as loading control. Amplification was performed for 40 cycles of 95° C. for 30 s, 60° C. for 1 min. Relative mRNA expression was calculated according to the δδCt-method.

Quantification of Cell Viability After Hypothermia-Rewarming Stress.

HUVEC were pre-treated with BN-80933, SUL-138 or SUL-150 (all 10 μM) for 1 hour in a humidified incubator at 37° C. Following pre-treatment, HUVEC were placed in a cold room (2-8° C.) for 24 hours and subsequently placed back at 37° C. for a 3 hour rewarming period. After 1 hour of rewarming, cell culture medium was replaced by prewarmed and filtered 0.4 mg/mL Neutral Red (#N463 8, Sigma-Aldrich) in culture medium. After the remaining 2 hour rewarming period, HUVEC were washed with PBS and NRU was solubilized in 100 μL resorption solution (50% ethanol, 1% ascetic acid in dH₂O). The absorbance was measured at a wavelength of 550 nm on a BioTek ELx808 plate reader. The recorded OD_{550 nm} values were normalized to control samples that were maintained at 37° C. at all times, which were assumed to be 100% viable.

Statistical Evaluation

All experiments were performed minimally in triplicate per condition. Data obtained from individual experiments were used for evaluation in GraphPad Prism 9.0 (GraphPad Software Inc, Ca). All datasets were normalized to vehicle controls. ANOVA followed by Bonferroni post hoc analysis was used to calculate statistical significance. Probability values (p) below 0.05 were considered significant.

Results

1. Endothelial NO-Production Following an Endotoxin (LPS) Challenge

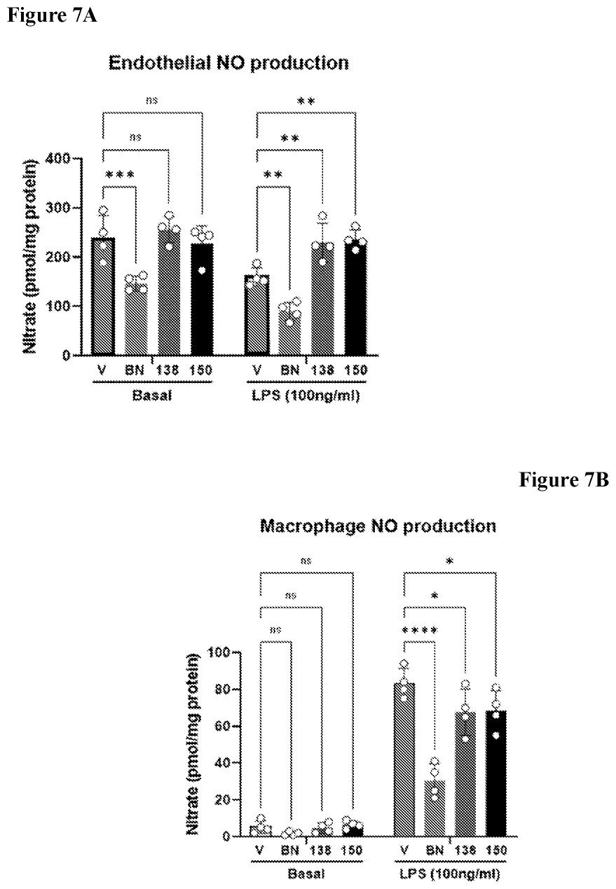

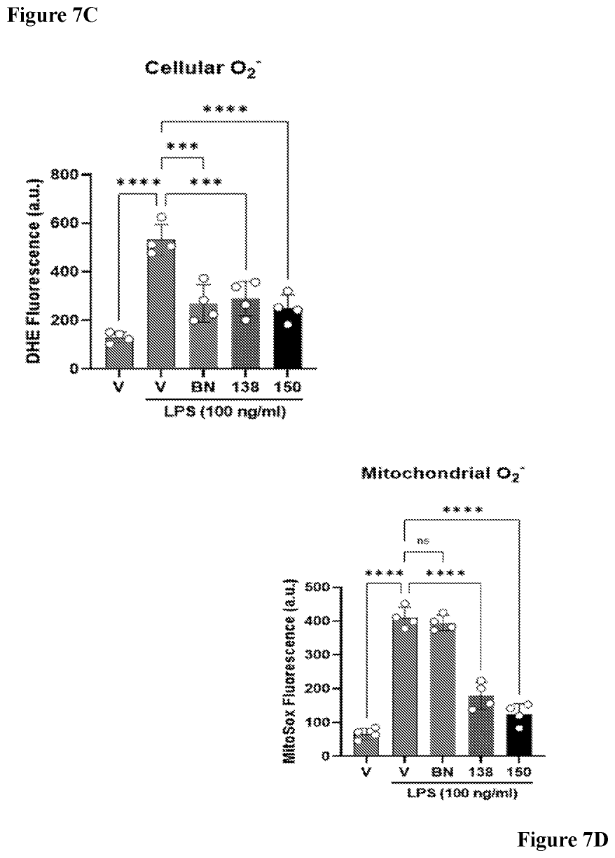

Basal endothelial NO production as measured by nitrate levels in the conditioned medium of HUVEC averaged at 239 pmol/mg protein (FIG. 7A). BN-80933 suppressed endothelial NO production by ˜38% (p<0.001), whereas SUL compounds do not change the basal NO production (FIG. 7A).

Endotoxins suppress endothelial NO production by limiting the activity of eNOS. HUVEC were pre-treated with vehicle (DMSO), BN-80933, SUL-138, or SUL-150 or (all 10 μM) prior to endotoxin (LPS 100 ng/ml) exposure for 24 hours. The nitrate levels in the conditioned medium from endothelial cells exposed to LPS averaged at 160 pmol/mg protein (˜33% reduction versus non LPS exposed HUVEC, p=0.002), which was lower than basal control levels. BN-80933 further suppressed the nitrate levels in the conditioned medium (FIG. 7A) to an average of 90 pmol/mg protein (p=0.008). In contrast, SUL-138 (p=0.008) and SUL-150 (p=0.004) mitigated the LPS-induced reduction in nitrate levels and their levels did not change from basal nitrate level.

2 Macrophage NO Production Following an Endotoxin (LPS) Challenge

Under physiological circumstances, very limited amount of NO is produced by inflammatory cells, yet under immunological stress, such as an endotoxin challenge, NO production via inducible nitric oxide synthase (iNOS) is boosted. Basal NO production as measured by nitrate levels in the conditioned medium of RAW264.7 macrophages was minimal, averaging 5 pmol/mg protein (FIG. 7B). Neither BN-80933, SUL-138, nor SUL-150 affected the basal production of extracellular nitrate.

LPS induces macrophage NO production by activating iNOS. RAW264.7 macrophages were pre-treated with vehicle (DMSO), BN-80933, SUL-138, or SUL-150 or (all 10 μM) prior to endotoxin (LPS 100 ng/ml) exposure for 24 hours. The nitrate levels in the conditioned medium from LPS exposed RAW264.7 macrophages averaged at 83 pmol/mg protein (˜16-fold increase versus non-exposed RAW264.7 cells, p=0.001). BN-80933 blunted the LPS-induced increase in extracellular nitrate level (p<0.001; FIG. 7B), consistent with its function as NOS inhibitor. Neither SUL-138 nor SUL-150 strongly suppressed the LPS-induced increase in nitrate production by RAW264.7 macrophages (FIG. 7B).

3 Cellular and Mitochondrial Superoxide Production

HUVEC produce a basal level of cytoplasmic (i.e. DHE) and mitochondrial (i.e. MitoSox) O₂⁻ fluorescence, which is greatly enhanced by LPS exposure (FIGS. 7C, 7D). Pre-treatment with BN-80933, SUL-138 and SUL-150 (all 10 μM for 30 min), mitigates the induction of cytoplasmic oxidative stress (FIG. 7C) with equal effectivity. SUL-138 and SUL-150 also precluded the LPS-induced increase in mitochondrial oxidative stress (FIG. 7C), which was not affected by the pre-treatment with BN-80933 (FIG. 7D).

These data show that BN-80933, SUL-138 and SUL-150 all harbor certain antioxidative capacity but the capacity differs, depending on e.g. cellular compartments.

4 Endothelial Inflammatory Activation

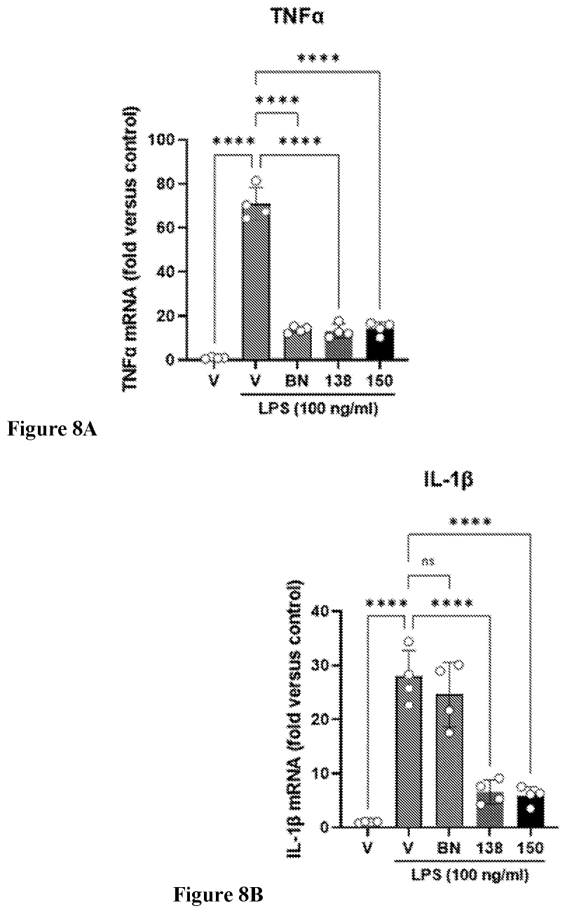

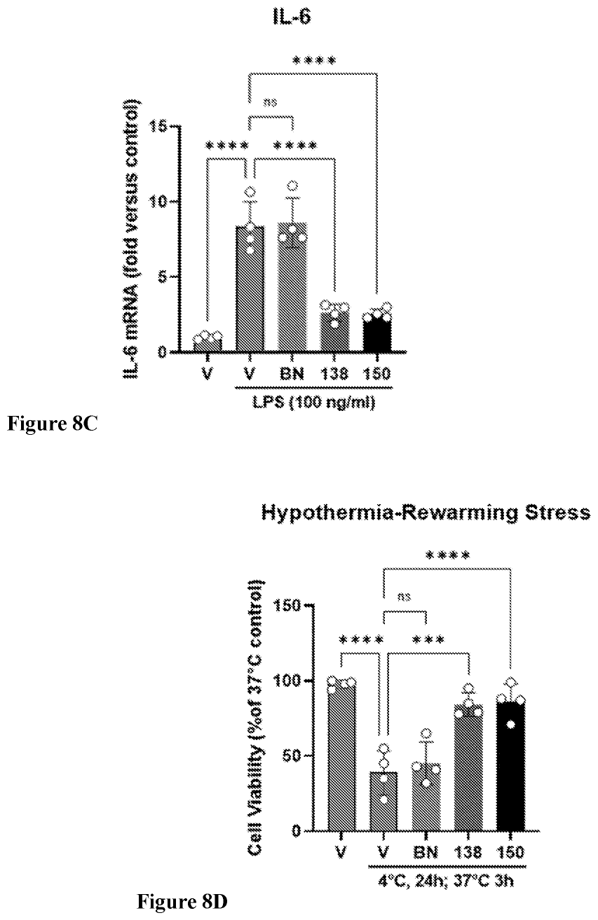

Endothelial cells have a low baseline gene expression for inflammatory cytokines, which is drastically increased upon exposure to endotoxin with varying magnitudes (FIG. 8A-C). Pre-treatment with BN-80933, SUL-138 and SUL-150 (all 10 μM for 30 min), precludes the induction of mRNA expression of the pro-inflammatory cytokine TNFα (FIG. 8A) with equal effectivity. Notably, pre-treatment of HUVEC with BN-80933 does not preclude the LPS-induced mRNA expression of the interleukins IL-1β and IL-6 (FIG. 8B, 8C), whereas SUL-138 and SUL-150 also precluded the LPS-induced increase in IL-1β and IL-6 (FIG. 8A-8C).

5 Endothelial Cell Viability After Hypothermia-Rewarming Stress

To confirm the different mechanism of action, endothelial cells were subjected to hypothermia as model for mitochondrial damage. Endothelial cells exposed to a cooling-rewarming cycle of 4° C. for 24 hours followed by 37° C. for 3 hours had a massive reduction in cell viability (˜61%) as compared to normothermic control cells (FIG. 8D). Pre-treatment with BN-80933 did not affect the hypothermia-associated reduction in cell viability (˜55% reduction), whereas both SUL-138 and SUL-150 maintained cell viability at a level just below the viability (˜15% reduction) of normothermic control cells. This shows that the mode of action of SUL-138 and SUL-150 is different from that of BN-80933.

Discussion

Reviewing the mechanism of action as investigated of BN-80933, SUL-138 and SUL-150 in the context of endotoxin-induced cell damage and hypothermia-rewarming stress, the following conclusions can be drawn:
1. BN-80933, but not SUL-138 or SUL-150, is a general inhibitor of nitric oxide synthase (NOS) enzymes in endothelial cells and macrophages.
2. BN-80933, SUL-138 and SUL-150 have cytoplasmic antioxidant capacity.
3. SUL-138 and SUL-150, but not BN-80933 have mitochondrial antioxidant capacity.
4. SUL-138 and SUL-150 preclude endotoxin-induced endothelial cell activation for all of TNFα; IL-1β and IL-6. BN-80933 inhibited TNFα only.
5. SUL-138 and SUL-150, but not BN-80933 preclude hypothermia-rewarming damage in endothelial cell.

Collectively, these data show that BN-80933 and SUL compounds have a vastly different mechanism of action and are active in distinct cellular compartments. BN80933 did have no activity towards mitochondria, in contrast to SUL compounds.

### Conclusions

The examples show SUL-138 to restore the xyphoid temperature after 24 hours after indication of CLP in mice. Treatment with SUL-138 decreases plasma levels of cytokines after sepsis induction. In particular, CLP-induced kidney dysfunction is precluded or significantly reduced by treatment with SUL-138 in mice as shown with the restoration of NGAL and urea function.

The examples also show that SUL-151 lowers mortality in septic Drosophila melanogaster and improves geotaxis at 24 h and 48 h after the induction of sepsis.

In septic endothelial cells, SUL-138 restores mitochondrial dysfunction.

BN-80933 and SUL compounds have a vastly different mechanism of action and are active in distinct cellular compartments. BN80933 did have no activity towards mitochondria, in contrast to SUL compounds.

Further in-vitro experiments show that a number of SUL compounds are active in an in-vitro model for sepsis, showing that the current findings are applicable to a group of compounds.

This whole body of in vivo and in vitro evidence shows that compounds as defined by the present invention show efficacy in treating sepsis, and organ disfunction caused by infection induced inflammation.

1. Compound according to formula (I) or (II), the hydroquinone analogue of formula (II), or a pharmaceutically acceptable salt thereof for use in the treatment or 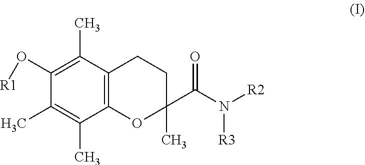
prophylaxis of sepsis; 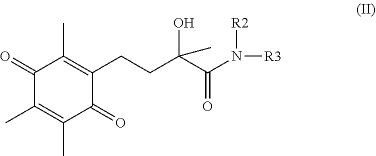
wherein R1 represents a hydrogen or prodrug moiety that can be removed in living tissue
and wherein either
i. R2 and R3 together with the N atom to which they are attached form a saturated or unsaturated, non-aromatic, optionally substituted 5-8 membered ring, having one to four N, O, or S atoms, wherein R2 and R3 together contain 3-12 carbon atoms;
ii. or R2 is a hydrogen atom, or an alkyl group with 1-6 carbon atoms, and R3 is an alkyl group, optionally substituted with nitrogen or oxygen, wherein the alkyl group comprises 3-12 carbon atoms, the alkyl group in R3 comprises one or more non-aromatic cyclic structures that may comprise nitrogen or oxygen atoms in the ring, and may contain linear and/or branched substituted groups, and one or more ethylenic unsaturations
iii. or R2 is a hydrogen atom, or an alkyl group with 1-6 carbon atoms, and R3 is an aryl group or arylalkyl group, optionally substituted with nitrogen or oxygen, wherein R3 comprises 6-14 carbon atoms, wherein R3 comprises one or more aromatic and/or non-aromatic cyclic structures that may comprise nitrogen or oxygen atoms in the ring, and may contain linear and/or branched substituted groups, and one or more ethylenic unsaturations, wherein the compound according formula (I) or formula (II) as free base has a molecular weight lower than 400 Da.

2. Compound for use according to claim 1, wherein R1 is hydrogen or forms together with the 6-oxygen an ester group with 2-6 carbon atoms.

3. Compound for use according to any one of claims 1-2, wherein the nitrogen can be amine, quaternary amine, guanidine, or imine and oxygen is hydroxyl, carbonyl or carboxylic acid; and/or oxygen and nitrogen together form amide, urea or carbamate groups.

4. Compound for use according to any one of claims 1-3, wherein in either compounds according to formula (I) or according to formula (II), R2 and R3 together with the N atom to which they are attached form a saturated ring incorporating an additional N atom, which ring is unsubstituted or substituted with an alcohol, or alkanol group having 1-4 carbon atoms.

5. Compound for use according to any one of claims 1-4, wherein the compound is a compound according to formula I.

6. Compound for use according to claim 5, wherein R2 and R3 together with the N atom to which they are attached form a 5-7 membered ring comprising one additional amine group, which ring is optionally substituted with methyl, ethyl, or alcohol substituted methyl or ethyl.

7. Compound for use according to any one of claims 1-3, wherein R2 is a hydrogen atom and R3 comprises a saturated cyclic structure having 4-7 carbon atoms and having one nitrogen atom, which ring may be substituted with an alkyl group, alcohol group, or with a group with 1-4 carbon atoms that may comprise an oxygen, carboxylic acid or amine group.

8. Compound for use according to claim 7, wherein the compound is a compound according to formula II and wherein R2 is a hydrogen atom and R3 comprises a cyclic structure having 4-6 carbon atoms and having one nitrogen atom which ring is optionally substituted with methyl, ethyl, or alcohol substituted methyl or ethyl.

9. Compound for use according to claim 1, wherein the compound is (6-hydroxy-2,5,7,8-tetramethylchroman-2yl)(piperazin-1-yl)methanone (SUL-121), ((S)-6-hydroxy-2,5,7,8-tetramethyl-N-((R)-piperidin-3-yl)chroman-2-carboxamide hydrochloride (SUL-13) or (6-hydroxy-2,5,7,8-tetramethylchroman-2-yl)(4-(2-hydroxyethyl)piperazin-1-yl)methanone (SUL-109) or a pharmaceutically acceptable salt thereof, as a racemic mixture or as one of its enantiomers.

10. Compound for use according to claim 9, wherein the compound is the S-enantiomer of SUL-109: S-(6-hydroxy-2,5,7,8-tetramethylchroman-2-yl)(4-(2-hydroxyethyl)piperazin-1-yl)methanone (SUL-138) or a pharmaceutically acceptable salt thereof.

11. Compound for use according to claim 9, wherein the compound is the S-enantiomer of SUL-121: S-(6-hydroxy-2,5,7,8-tetramethylchroman-2yl)(piperazin-1-yl)methanone (SUL-151) or a pharmaceutically acceptable salt thereof.

12. Compound for use according to any of claims 1-8, wherein the compound according formula (I) or formula (II) as defined in options (i) and (ii) has a molecular weight lower than 500 Da, preferably lower than 450 Da and most preferred lower than 400 Da.

13. Compound for use according to any of the preceding claims, wherein the use is for the treatment or prophylaxis of organ dysfunction caused by a dysregulated host response to infection.

14. Compound for use according to claim 13, wherein the organ is one or more of lung, heart and blood vessels, liver, kidney, brain, or intestines, preferably kidney.

15. Compound for use according to any one of claims 1-14, wherein the treatment or prophylaxis is done in a combination therapy with one or more common measures to treat sepsis.
